(19) United States
(12) Patent Application Publication
(10) Pub. No.: US 2023/0052080 A1
(43) Pub. Date: Feb. 16, 2023

(54) APPLICATION OF DEEP LEARNING FOR INFERRING PROBABILITY DISTRIBUTION WITH LIMITED OBSERVATIONS

(71) Applicant: International Business Machines Corporation, Armonk, NY (US)

(72) Inventors: Shangying Wang, San Jose, CA (US); Simone Bianco, San Francisco, CA (US)

(21) Appl. No.: 17/398,996

(22) Filed: Aug. 10, 2021

(51) Int. Cl. G16B 40/00; G06N 3/04; G16B 20/00

(52) U.S. Cl. CPC G16B 40/00; G06N 3/0445; G16B 20/00

(57) ABSTRACT

A method for application of a deep learning neural network (NN) for predicting the probability distribution of a biological phenotype does not require any assumption or prior knowledge of the probability distributions. The NN may be a recurrent neural network (RNN) or a long short-term memory (LSTM) network. The NN includes a loss function, which is trained on limited observations, as low as one observation, which is obtained from a large data set related to a biological system. The NN with the trained loss function is capable of calculating if readings that are outside of the mean for the data set are inherent to the biological system or are outlier readings. The output of the method is a continuous probability distribution of the biological phenotypes for each input parameter or set of parameters from the biological data set.

### TECHNICAL FIELD

The present invention relates generally to the application of artificial intelligence to predict the phenotype of biological systems and more specifically to the application of deep learning neural networks to predict phenotypic probability distributions in biological systems from limited observations.

### BACKGROUND OF THE INVENTION

Biological systems are inherently stochastic due to the presence of both extrinsic noise, which is due to fluctuations of the environment, and intrinsic noise, the latter of which produces variations in identically regulated quantities within a single cell. For example, genetically identical cells in identical environments can display variable phenotypes. The intrinsic noise in biological systems causes difficulties in the ability to detect, combat, and categorize biological and/or clinical data. Biological intrinsic noise also hinders the ability to understand the relationship between underlying genetic/environmental conditions and phenotypic observations. Intrinsic noise has been shown to play a crucial role in gene regulation mechanisms; thus, predicting only an average value of outputs is not sufficient for the study of the dynamics of biological systems.

Currently, the most common approach to overcome the intrinsic noise in biological systems is to perform more measurements. This time-consuming and expensive approach is not sustainable for complex biological systems where there are many varying parameters and the feasibility of obtaining sufficient observations for all possible input combinations is very low.

### SUMMARY OF THE INVENTION

In one aspect, the present invention relates to a method of predicting probability distribution of a biological phenotype comprising: gathering a data set comprising at least 3000 input parameters for a biological system and generating a limited data set comprising 1-10 output observations through experimentation and/or simulation of the input parameter data set; building a deep learning neural network comprising a loss function and training the loss function with the limited data set of output observations; and training the neural network with the input parameter data set, wherein output from the trained neural network comprises a predicted probability distribution of a biological phenotype associated with the biological system.

In another aspect, the present invention relates to a method of predicting probability distribution of a biological phenotype comprising: gathering a data set comprising at least 3000 input parameters for a biological system and generating a limited data set comprising 1-10 observations through experimentation and/or simulation of the input parameter data set; building a recurrent neural network (RNN) comprising a negative log-likelihood loss function and training the negative log-likelihood loss function with the limited data set of output observations; and training the RNN with the input parameter data set, wherein output from the trained RNN comprises a predicted probability distribution of a biological phenotype associated with the biological system.

In a further aspect, the present invention relates to a method of predicting probability distribution of a biological phenotype comprising: gathering a data set comprising at least 3000 input parameters for a biological system and generating a limited data set comprising 1-10 observations through experimentation and/or simulation of the large data set; building a long short-term memory (LSTM) network comprising a negative log-likelihood loss function and training the negative log-likelihood loss function with the limited data set of output observations; and training the LSTM network with the input parameter data set, wherein output from the trained LSTM network comprises a predicted probability distribution of a biological phenotype associated with the biological system.

Additional aspects and/or embodiments of the invention will be provided, without limitation, in the detailed description of the invention that is set forth below.

### BRIEF DESCRIPTION OF THE DRAWINGS

FIG. 1 is a diagram showing the model structure for the deep learning recurrent neural network (RNN) described herein, which is integrated with a negative log-likelihood loss function and which predicts probability distributions with limited observations.

FIG. 2 is a diagram showing the training of a long short-term memory (LSTM) algorithm, which may be used to run an RNN as described herein.

FIGS. 3A and 3B are diagrams showing the algorithm architecture for a LSTM. FIG. 3A shows the general LSTM architecture and FIG. 3B shows the algorithm architecture of a single LSTM cell.

FIG. 4 shows the full structure of an LSTM network.

FIG. 5 is a diagram of the two-state model for stochastic gene transcription in single cells.

FIG. 6 are graphs showing theoretical probability distribution curves for mRNA numbers in a sample that may be obtained through application of the deep learning neural network described herein.

FIGS. 7A-7H are graphs showing probability distribution curves for mRNA numbers in a sample where the deep learning neural network is trained with n=1 (FIG. 7A), n=3 (FIG. 7C), n=10 (FIG. 7E), and n=100 (FIG. 7G) observations and the respective trained neural networks are applied to test sets (FIGS. 7B, 7D, 7F, 7H)

FIGS. 8A and 8B are graphs that compare mRNA predicted means calculated with the deep learning neural network described herein (right panels) against mRNA sample means not run through a neural network (left panels) for n=1 (FIG. 8A, top graphs), n=3 (FIG. 8A, bottom graphs), n=10 (FIG. 8B, top graphs), and n=100 (FIG. 8B, bottom graphs) observations.

FIG. 9 are graphs that compare the accuracy of the deep learning neural network, calculated with a negative log-likelihood loss function, against two control tests, a root mean square error (RMSE) test and a coefficient of determination (R²) test.

### DETAILED DESCRIPTION OF THE INVENTION

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

As used herein, the term “neural network” refers to an artificial intelligence computing system that is inspired by the biological neural networks of animal brains. Neural networks include a collection of connected units or nodes (also known as neurons), which can transmit signals (in the form of numbers) to other nodes. The connections between the nodes are called edges. Together, nodes and edges have a weight that adjusts as the learning proceeds where the weight increases or decreases with the strength of the signal at a connection. Where nodes have a threshold, a signal is sent only if the aggregate signal crosses the threshold. When a node receives a signal, it processes the signal and outputs the signal to other nodes to which it is connected.

Neural networks are trained by processing examples that contain a known input and result, forming probability-weighted associations between the input and the output (i.e., the result) and storing the trained information within the data structure of the network. The training of a neural network from a given example is conducted by determining the difference (i.e., the error) between the output processed from the network (typically a prediction) and a target output. In response to the error, the network adjusts its weighted associations according to a learning rule and the error value. Successive adjustments result in the neural network producing output that is increasingly similar to the target output.

As is known to those of skill in the art, neural network learning may be supervised, unsupervised, self-supervised, or semi-supervised. With supervised learning, labeled datasets are used to train the neural network algorithms. With unsupervised learning, the neural network trains itself with unlabeled data by recognizing patterns that solve clustering or association problems. With semi-supervised learning, the neural network is trained with a small amount of labeled data and a large amount of unlabeled data. With self-supervised learning, the neural network recognizes patterns in unlabeled data, which is subsequently self-labeled and used on downstream operations.

As used herein, the term “deep learning” refers to a neural network with multiple layers between the input and output layers. In deep learning, each level learns to transform its input data into slightly more abstract and composite representations. The “deep” in deep learning refers to the number of layers through which the data is transformed. A deep learning neural network is capable of disentangling abstractions within the multiple layers of the network to identify features that require improved performance. All deep learning algorithms may be supervised, unsupervised, semi-supervised, or self-supervised.

As used herein, the term “recurrent neural network” (RNN) refers to a deep learning neural network that that allows previous outputs to be used as inputs while having hidden states. RNNs differ from traditional feed forward neural networks, the latter of which move in only one forward direction from the input nodes through hidden nodes to the output nodes with no cycles of loops in the nodes. With RNNs, connections between nodes form a directed graph along a temporal sequence thus allowing the RNN to use their memory to process variable length sequences of inputs; in this way, RNNs exhibit temporal dynamic behavior. RNNs are capable of processing inputs of any length without causing an increase in the model size. Further, the input weights within the model are shared across time. Like all neural networks, RNNs can be supervised, unsupervised, semi-supervised, or self-supervised.

As used herein, the term “loss function” refers to a negative log-likelihood correction undertaken by the RNN at each time step, which is represented by Formula (1):

$$loss function = {\sum }_{i} - log ( P \frac{{o}_{i}}{{x}_{i}} \vert ) \qquad ( 1 )$$

where o is the number of output observations, X represents the parameters; i is the unknown size of the sample; and P is the probability function of the observations to the parameters. The loss function is trained using a limited set of collected observations in order to remove the uncertainties that are inherent in an RNN. The purpose of the loss function is to maximize the probability that the observed data is within the predicted probability distribution of the RNN. The loss function does this by calculating the sum of all of the probabilities of the observed data within each parameter. Because the loss function is a negative log-likelihood, its integration into the RNN minimizes the loss of observations that would otherwise fall outside of a typical mean analysis. The first step in the training of the RNN described herein is the establishment of the loss function. During training of the RNN, the loss function compares the prediction outcomes to the desired output resulting in output values throughout the time series and propagation of the loss function back through the RNN to update the input weights; thus, every node that has participated in the calculation of the output associated with the loss function has its weight updated to minimize the error throughout the RNN.

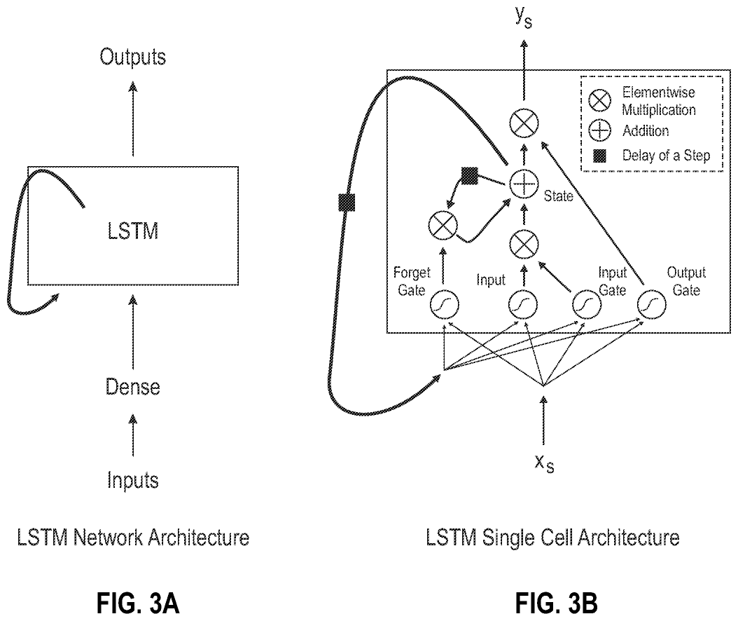

As used herein, the term “long short-term memory” (LSTM) refers to an RNN architecture where the neural network is capable of learning order dependence in sequence prediction problems. An LSTM network can classify, process, and make predictions based on time series data to avoid the lags of unknown duration between important events in a time series. In this way, LSTMs can overcome the vanishing gradient problem that is encountered when training RNNs. The learning process of LSTMs is typically self-supervised. FIG. 3A shows the algorithm architecture of an LSTM cell. As shown therein, the neural network comprises an input layer that is connected to a fully connected dense layer, where the output from the fully connected dense layer is fed to an LSTM cell. As shown in FIG. 3B, the LSTM cell manages two state vectors: one responsible for short-term memory (the external loop) and the other responsible for long-term memory (the internal self-loop).

As used herein, the term “probability distribution” refers generally to a statistical function that describes all the possible values and likelihoods that a random variable can take within a given range. A probability distribution (also referred to in the art as a probability distribution function or pdf) is used when a set of probabilities is treated as a unit. Within the context of probability distributions, there are two types of data: (i) discrete data, which has specific values, such as 1, 2, 3, 4, 5, 6, etc., but not 1.5 or 2.75; and (ii) continuous data, which can have any value within a given range and which can be finite or infinite. Probability distributions generally require assumptions regarding the data within the distributions; the present invention does not require any assumptions regarding the input data or the probability distributions.

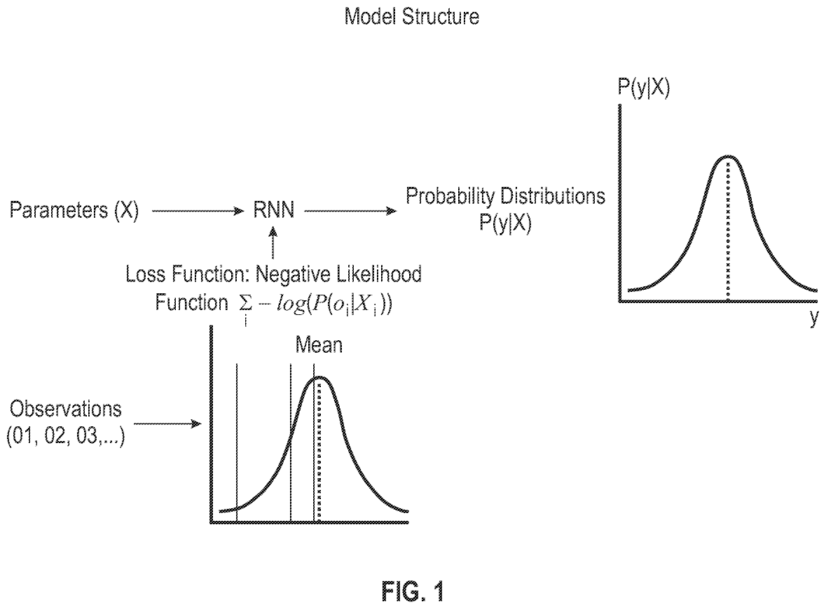

The deep learning model described herein predicts the probability distribution of phenotypic observations (y) within a biological population with at least one observation for each input parameter (X) without any assumptions or prior knowledge of the probability distributions. The deep learning model learns the probability distribution of the observations p(y/X) directly from the data. The deep learning model has the capacity to explore unknown biological systems and can facilitate quantitative understanding of biological systems, such as cellular systems and biological collectives, and provide the tools necessary for the design of synthetic gene circuits. FIG. 1 is a diagram showing the deep learning model structure with a RNN and a negative log-likelihood loss function. As explained above, the loss function is trained with a limited number of observations (e.g., 1, 2, 3, or 10). Example 1 provides an outline of the process for building the deep learning probability distribution model described herein.

Within the context of typical deep learning models, intrinsic noise in biological systems results in deep learning models that are only able to predict a determined phenotype, or a set of determined phenotypes, per genetic and/or environmental condition. A deficiency of deep learning predictors known in the art is the general inability to identify a given observation as an outlier relative to the training data for the model. A naïve deep learning classifier will make a prediction based on the mean of all available candidates; this is problematic in the case of stochastic biological processes where the mean value does not represent the dynamics of the entire biological process. Intrinsic noise thus makes the mapping of input parameters (e.g., genotype and/or environmental factors) to output observations (e.g., noisy phenotypic observations) difficult. Due to intrinsic stochasticity present in many biological processes, observations that are far away from the population mean results in predictive models that are hard to build, and after building, predictions that do not provide meaningful information. The present invention is an insightful prediction model for intrinsically noisy/stochastic biological systems that provides a complete probability distribution of genetic variations based on limited phenotypic observations.

With synthetic biology systems, the number of parameters that can affect observations is often very large; consequently, optimal design of synthetic biology systems is subject to high uncertainty. The deep learning model described herein can be applied to biological systems, including synthetic biology systems, to reduce the time and computational cost for simulations and experiments required to predict biological objectives and optimize synthetic design. The ability to infer probability distributions based on limited observations, including a single observation, in the context of intrinsically noisy/stochastic biological systems is beneficial for biological system design and optimization.

The ability of the probability distribution method described herein to reduce noise has many advantages. For example, by reducing the noise inherent in biological systems, the method may be used to reliably predict the average values of a population. Further, by reducing the noise in each observation, the method improves the performance of predictive models that map from continuous and varying parameters. By training the negative log loss function, the method also has the capacity to estimate the noise for each input combination.

The probability distribution prediction method may be applied to the design of a single biological system, such as a cell, or a collective biological system, such as a microbial colony. For example, in one embodiment, the probability distribution method may be applied to the design of a single cell whose input is too large to explore experimentally through genetic, physical, and/or environmental modifications and whose output is a desired phenotype. In another embodiment, the probability distribution method may be applied to predict the biological functions of a microbial colony, such as antibiotic resistance and duplication rate, from various input growth conditions, such as nutrient concentration, pH, and temperature.

Examples of biological system input parameters that may be used with the probability distribution prediction method described herein include, without limitation, cell growth rate, cell lysis rate, cell motility, gene expression, nutrient concentration, temperature, pH, activation rate, transcription rate, temperature, agar density, and combinations thereof. Examples of biological system output is a phenotype selected from the group consisting of number of mRNA produced, number of amino acids, number of proteins, cellular growth, cellular adhesion, cellular sensing, fluorescence strength, optical density, chemical concentration, and combinations thereof.

In application, if the probability distribution within a biological system is for a discrete variable (such as the number of mRNAs, amino acids, and/or proteins), the sum of the probability for all possible numbers will be one. By contrast, if the probability distribution with the biological system is for a continuous variable (such as the strength of a fluorescent or optical density, or the concentration of chemicals), the variable first needs to be discretized and then the total area under the probability density function will be equal to one. A priori knowledge of the shape of the probability distribution is not required.

In one embodiment, the deep learning neural network is an RNN comprising a loss function, the latter of which is used to minimize the uncertainty of the RNN during training. By minimizing the loss function, the probability distribution is continuous and thus there is no abrupt change of the probability distribution when varying the input parameters.

In another embodiment, a probability distribution of a stochastic biological system is predicted by carrying out the following actions: (i) data preparation comprising a large number of different input parameter values that are chosen for a biological system, where, for each parameters set, a limited number of observations (as low as one) are collected for the biological system; (ii) training an algorithm based upon an RNN using the input parameter values, where the input layer of the RNN is composed of the input conditions, the output layer of the RNN is a probability distribution function, and the nodes of the RNN are initialized to random values; (iii) applying a loss function to the RNN to minimize uncertainty during training, where the loss function is trained with the limited number of collected observations; and (iv) the output probability distribution function is initialized to a uniform distribution and modified at each training epoch to minimize the loss function, where the gradient is clipped to prevent exploration.

Where the observations are discrete variables (e.g., mRNA counts, protein counts, etc.), the RNN predicts the probability distribution value for all possible discrete numbers and one or more additional neural network layers are necessary to ensure that the sum of the predicted probability distribution for an input condition equals one. If the observations are continuous variables (e.g., concentrations, optical density, fluorescence, etc.), the RNN provides predictions of the probability distribution value by interpolating discrete observations where the last layer is normalized with a normalization factor to ensure that the cumulative trapezoidal numerical integration of the probability distribution equals one.

In another embodiment, the deep learning neural network comprises an LSTM network. The prediction of probability distribution using an LSTM network first requires the identification of a large number of distinct parameter sets (e.g., 10,000). For each parameter set, a limited set of observations (e.g., 1, 2, or 3) are sampled and collected and used for training of the loss function, which is used to minimize the uncertainty during the training of the LSTM network. Where there are no specific biological boundaries, prior to the training of the LSTM, the range of the probability distribution is set such that for all possible parameter sets, the predicted probability distribution will not fall out of the set range. Where L is the value of the largest observation, the edge of the distribution M is calculated according to Formula (2):

M=2*L. (2)

For biological systems, the lower bound of the distribution is zero.

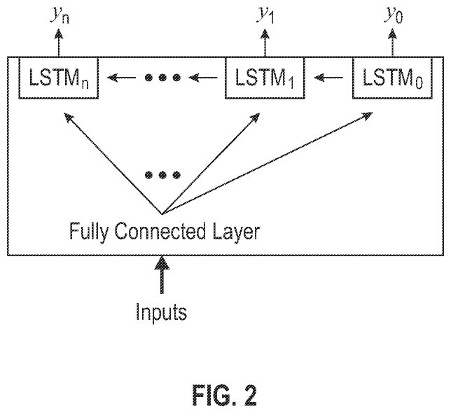

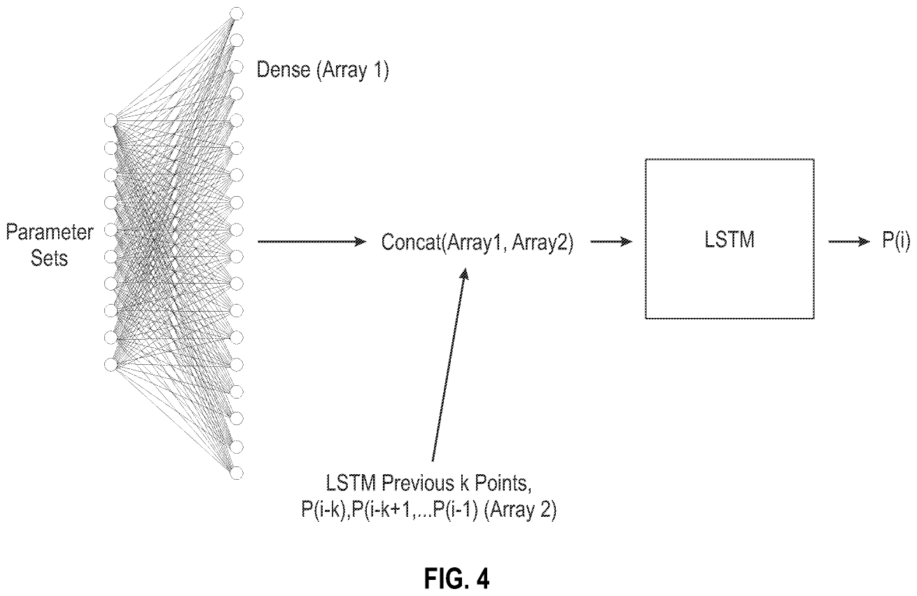

With reference to FIG. 2, the LSTM is trained with input system parameters where the input layer is connected to a fully connected dense layer and output from the fully connected layer is fed into the LSTM cells (LSTM₀, LSTM₁, . . . LSTMₙ) where probability distribution outputs are identified for different biological phenotypes (y₀, y₁, . . . yₙ). FIG. 3A shows the general architecture for the probability distribution model described herein implemented with an LSTM algorithm. FIG. 3B shows the LSTM single cell architecture, including the LSTM external short term memory loop (also shown in FIG. 3A) and the internal long-term memory loop. Where the probability distribution consists of n points, P(0), P(M/(n−1)), P(2*M(n−1)), . . . P((n−2)*(M/(n−1)), and P(M), where n is sufficiently large to ensure smoothness of the probability distribution (e.g., 500), the probability distribution prediction of an LSTM network is a [P(0), P(M)] array, which is shown schematically in FIG. 4. With continued reference to FIG. 4, in order to predict the probability value for the ith point, where 0<i<n, the LSTM array must be concatenated from the output of the fully connected layer (array 1) with the LSTM output of the previous k points: i−k, i−k+1, . . . i−1 (array 2 with k elements), where k is a hyperparameter to be tuned to optimize the performance of the neural network. The concatenated array is used as the input for an LSTM cell for predicting the probability value of the ith point. If i≤k, the missing value for i in array 2 is filled with a constant value, such as, for example, 0 or 0.5. During the training, the output of the LSTM network (i.e., the probability distribution) is updated at each training epoch to minimize the loss function.

In a further embodiment, the loss function for both the RNN and the LSTM network is a negative log-likelihood as defined herein. In another embodiment, the output of both the RNN and the LSTM network is a continuous probability distribution for each input parameter or parameter set.

For purposes of illustration, the following discussion will be directed to the use of the deep learning neural network described herein for predicting the probability distribution of mRNA in a sample. It is to be understood that the application of the deep learning neural network to predict the probability distribution of mRNA in a sample is exemplary is not intended to limit the application of the deep learning neural network to other applications. As is known to those of skill in the art, transcription and translation are the two main steps of gene expression. A gene is first transcribed into mRNA by an RNA polymerase enzyme and then the mRNA is translated into proteins. Gene expression is intrinsically stochastic due to the inherent randomness within gene expression. While the stochasticity in gene expression has the advantage of advancing diversity of a species, it introduces uncertainty in theoretical modeling.

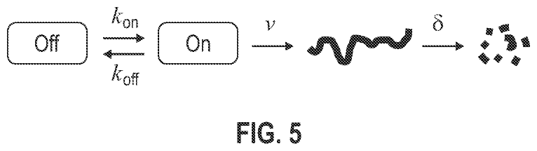

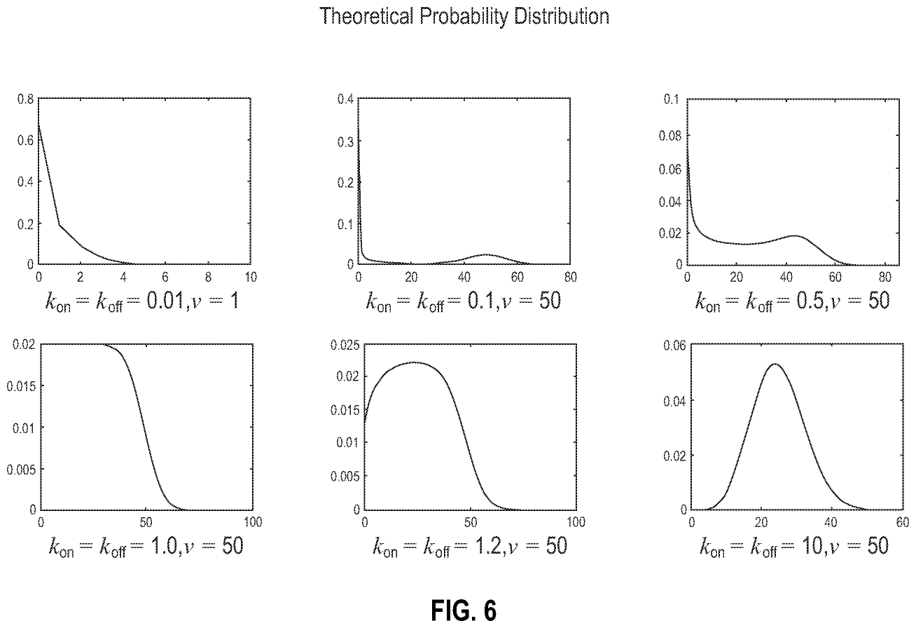

FIG. 5 provides a schematic diagram of the two-state model for stochastic gene transcription in single cells, the latter of which includes bacteria, yeast, and mammalian cells. The diagram in FIG. 5 indicates that a gene promoter transits between inactive (gene off) and active (gene on) where Kₒₙ represents a gene in an activated state; K_{off} represents a gene in an inactivated state; ν is the transcription rate; and δ is the mRNA degradation rate. Example 2 describes the application of the deep learning neural network described herein to predict the probability distribution of mRNA numbers in a sample and FIG. 6 presents graphs showing the theoretical probability distributions for transcription for different values of Kₒₙ, K_{off}, and ν, where Kₒₙ=K_{off}, ν>1, and δ=1. Unlike prior art probability distribution predictions that require assumptions and have expected curves, the graphs in FIG. 6 were obtained without any assumptions regarding gene transcription probability distributions and hence, no particular curve shape was expected at the beginning of the theoretical analysis. Because no expected curve is expected from the deep learning neural network, the resultant curves may resemble any probability distribution curve known in the art. For example, in FIG. 6, the six graphs have curves that resemble the following known probability distribution curves: (1) an exponential decay curve; (2) a bimodal distribution curve; (3) a bimodal distribution curve; (4) a Poisson or step function curve; (5) a curve between a Gaussian and Poisson curve; and (6) a Gaussian curve.

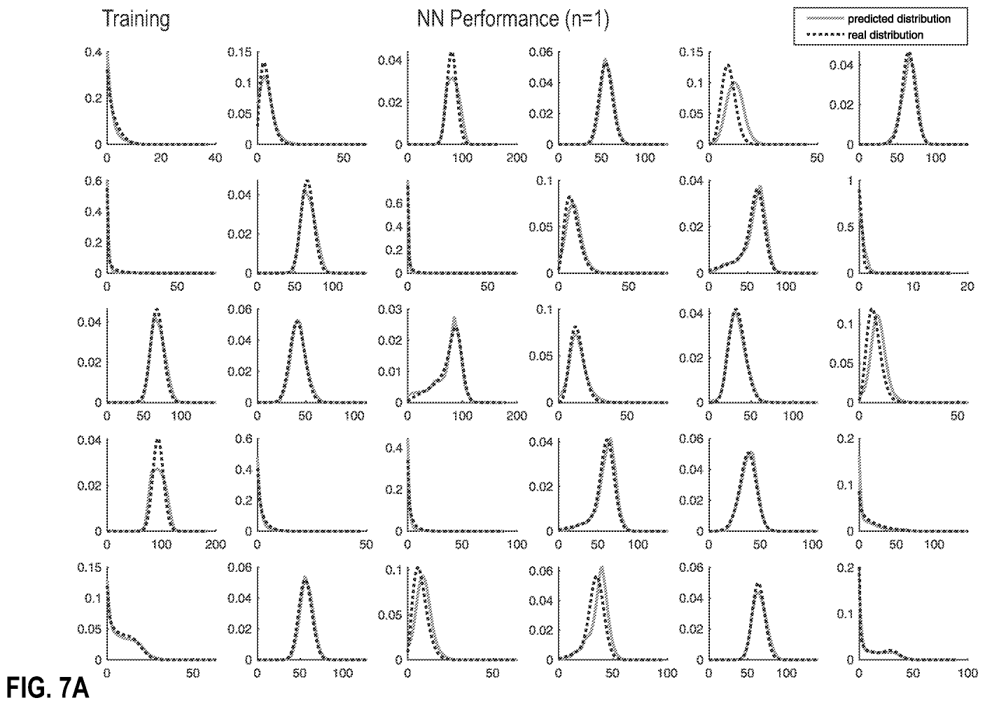

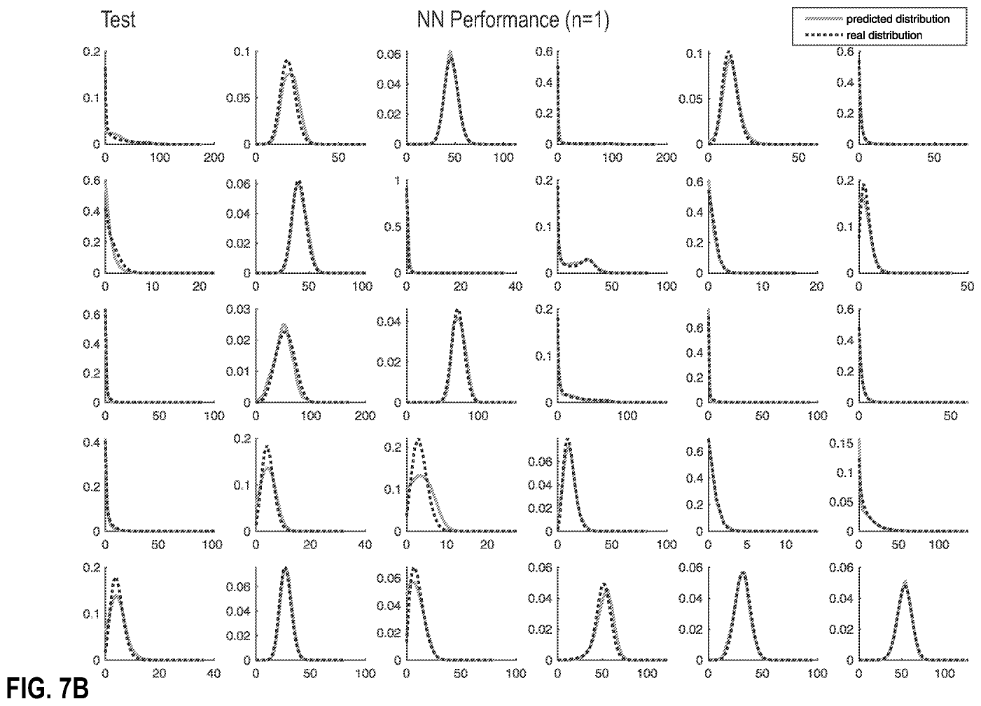

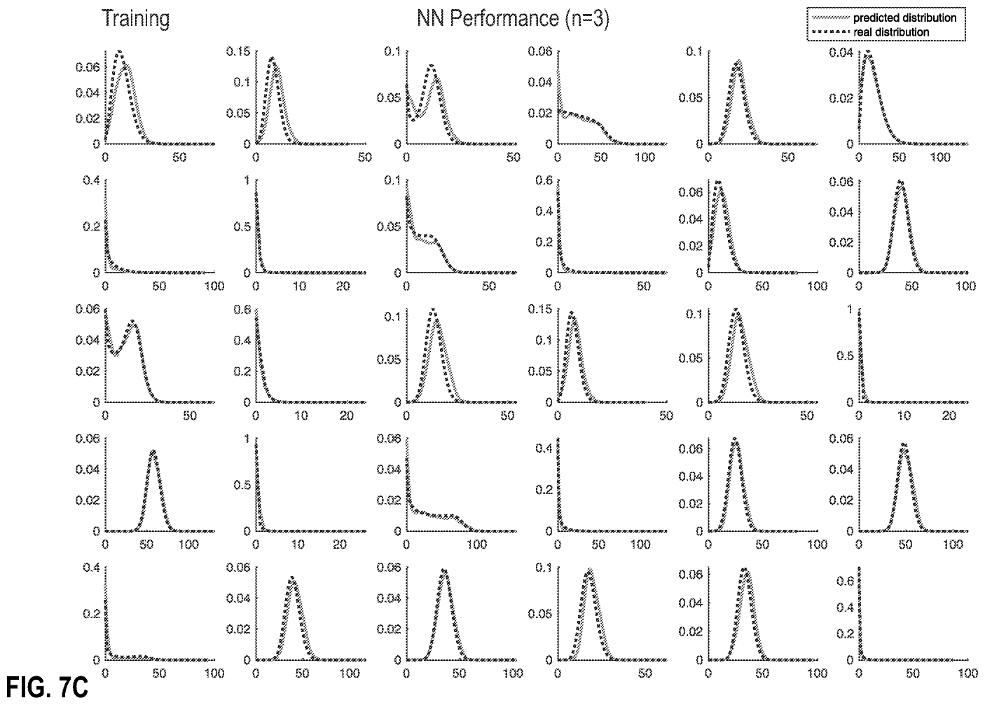

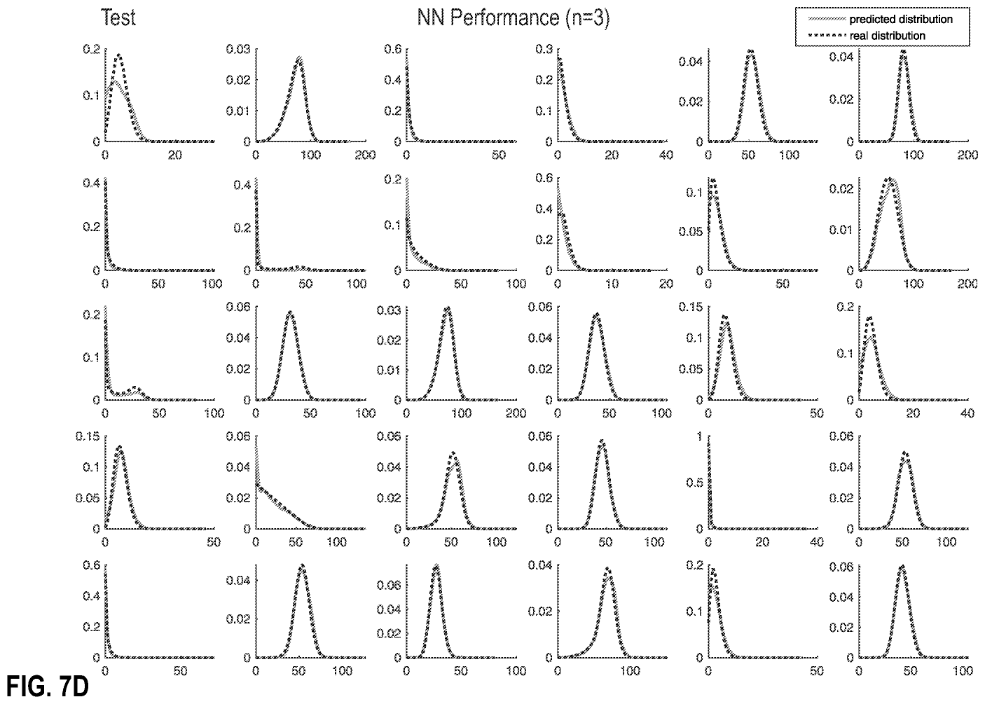

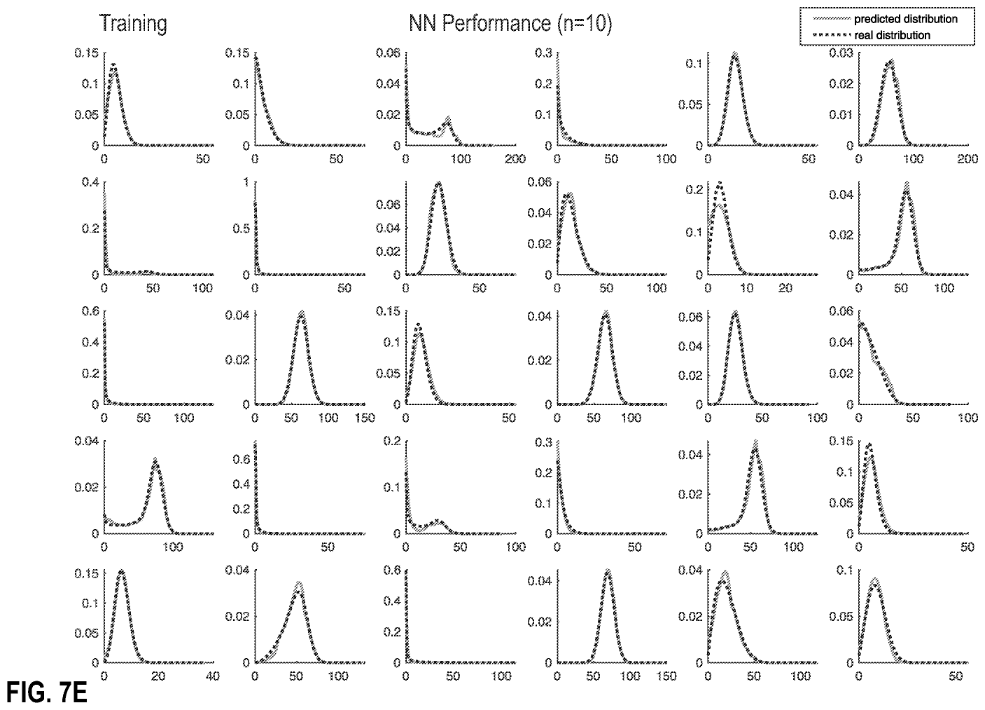

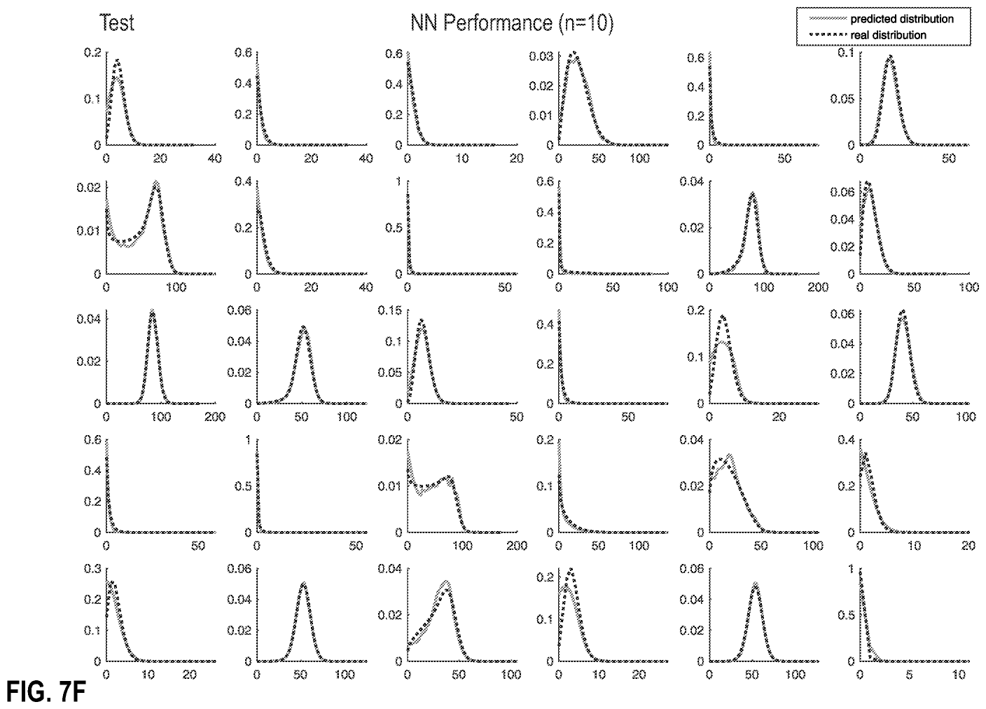

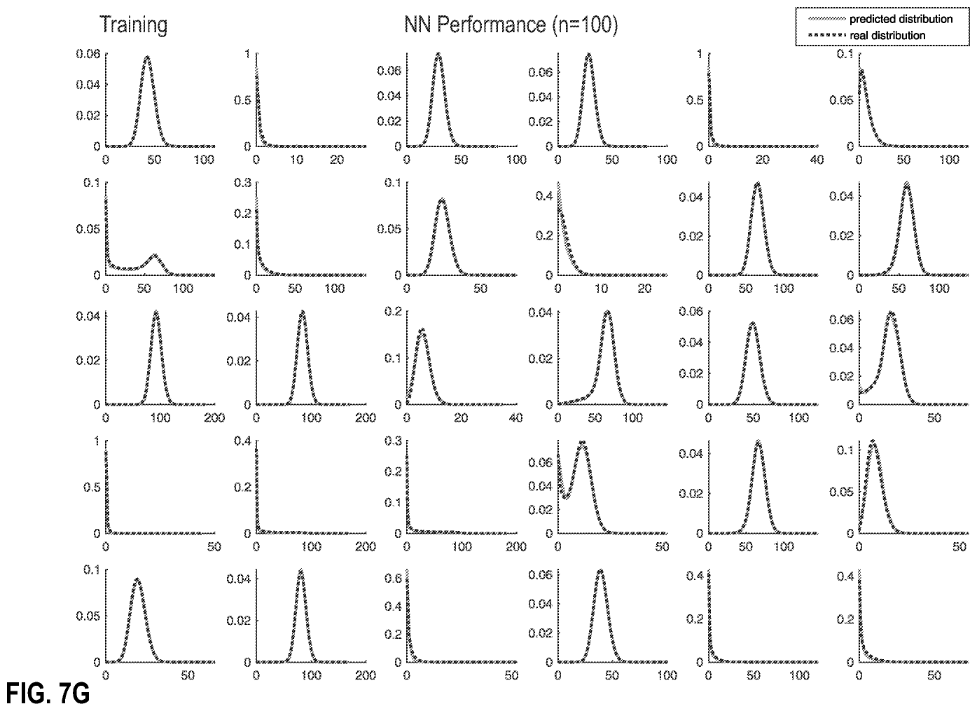

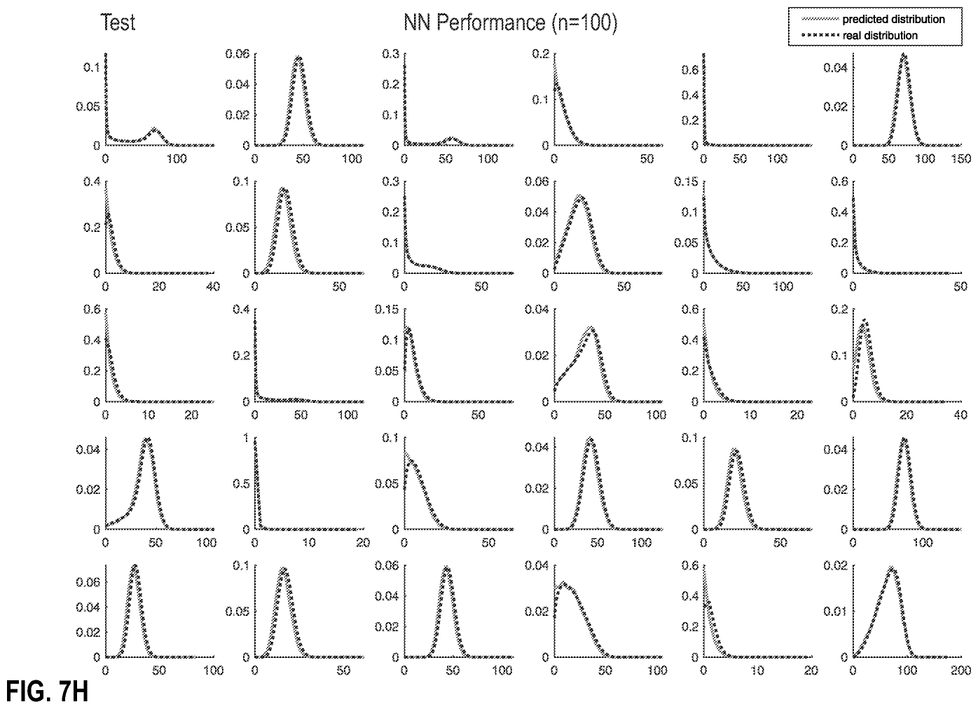

Example 3 describes application of the deep learning neural network described herein to predict the probability distribution of the number of mRNA in a sample comprising a 2000 test data set where 1, 3, 10, and 100 output phenotypic observations (n=1, 3, 10, and 100) are used to train the NN within a training data set of 10000 data points. To determine the number of mRNA, the Kₒₙ, K_{off}, ν, and δ parameters as described above were used where Kₒₙ=K_{off}, ν>1, and δ=1. As shown in FIGS. 7A-7H, the neural network probability distribution performance for the 2000 data point test set is comparable for all four limited observations, n=1 (FIGS. 7A, 7B), n=3 (FIGS. 7C, 7D), n=10 (FIGS. 7E, 7F), and n=100 (FIGS. 7G, 7H). As would be expected, the training set for the n=100 experimental data set showed predicted distribution training data points that were almost identical to the real distribution training data points with the training showing a lesser degree of overlap, albeit not by much, with the smaller number of training observations. Nevertheless, when the test data was applied to the trained neural network, the predicted distribution of the test data was very close to the real distribution for all four observations, with the n=100 observation showing just a slight increase in accuracy (which would be expected due to the higher number of training observations). The results shown in FIGS. 7A-7H surprisingly and unexpectedly demonstrate that limited observations, as low as a single observation, may be used to successfully train the deep learning neural network described herein.

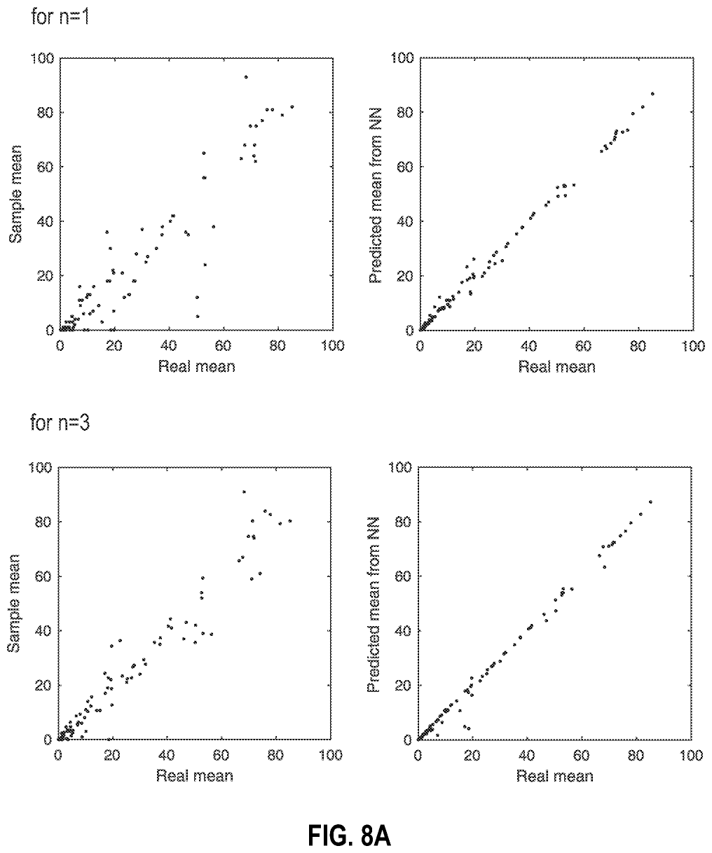

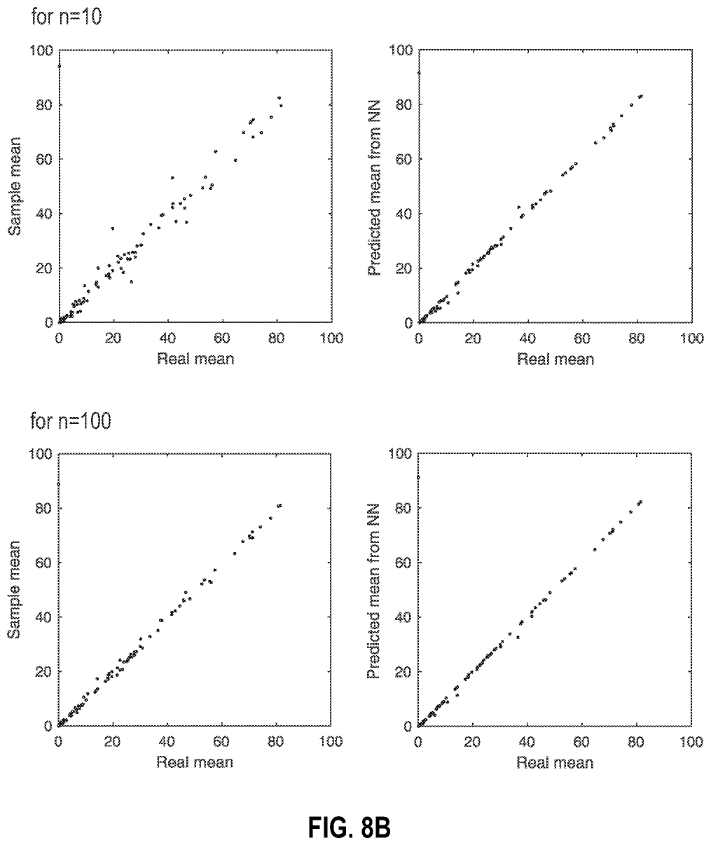

Example 4 addresses the ability of the probability distribution predictions of the deep learning neural network to overcome the intrinsic noise present in gene transcription. As shown in FIGS. 8A and 8B, the predicted values for the mRNA probability distributions obtained with the n=1, 3, 10, and 100 observations show little to no noise in comparison to sample mean values calculated with the same number of observations. The sample mean graphs in FIGS. 8A and 8B show a great deal of noise with the n=1, 3, and 10 observations whereas the comparable predicted mean graphs show very little noise. The results of this noise experiment surprisingly and unexpectedly demonstrate that even with a single observation (FIG. 8A, top left panel), the neural network with the negative log-likelihood loss function produces accurate probability distribution predictions for the number of mRNA in a sample.

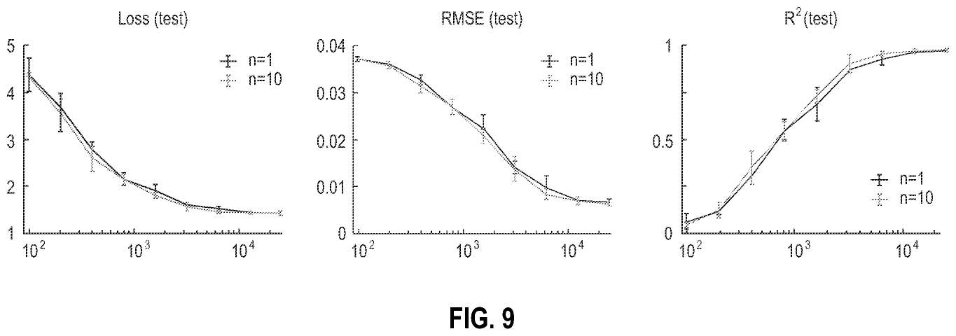

Example 5 addresses if the size of the neural network training data set affects the accuracy of the mRNA probability distribution predictions. Using two observation sets, n=1 and n=10, and a fixed size test data set of 3000, the training of the deep learning neural network with a negative log-likelihood loss function was carried out with nine different input parameter training set sizes: 100, 200, 400, 800, 1600, 3200, 6400, 12800, and 256000. FIG. 9 shows that the n=1 observations are comparable to the n=10 observations for all training data sets and that the loss of observed data is reduced as the training data set increases. The loss of data for the negative log-likelihood loss function test shows good accuracy at 3200 and 6400 input parameters and shows no discernable difference in accuracy between the 12800 and 256000 input parameters. The data in FIG. 9 demonstrates that in one embodiment, the claimed deep learning neural network can generate a limited data set of 1-10 output observations from a training data set of at least 3000 input parameters. In another embodiment, the deep learning neural network generates output observations from the input parameter training set in the range of, for example, 1-10, 1-5, or 1-3 output observations, regardless of the size of the training set. In a further embodiment, the deep learning neural network generates a single output observation from the input parameter training set, regardless of the size of the training set. In another embodiment, the training data set has at least 5000 input parameters. In a further embodiment, the training data set has at least 10,000 input parameters. In another embodiment, based upon the type of data being analyzed, the number of input parameters, n, may range from n<1<infinity, the only limitation on the upper range of the input parameters being the capability of the computers that are running the analysis. For example, it would be within the scope of the deep learning neural network described herein to process training data sets that have as much as 100,000, 1 million, 10 million, 100 million, or more data points.

With continued reference to FIG. 9, the two control tests, a root mean square error test (RMSE) and a coefficient of determination (R²) test, both appear to plateau in accuracy for n=10 at a training test size of 6400 and for n=1 at a training test size of 12800. The data is FIG. 9 shows that the integration of a negative log-likelihood loss function into the deep learning neural network described herein reduces the need for large training data sets.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, a graphics processing unit (GPU), programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

### EXPERIMENTAL

The following examples are set forth to provide those of ordinary skill in the art with a complete disclosure of how to make and use the aspects and embodiments of the invention as set forth herein. The Examples that follow were performed using RNN and/or LSTM networks. To cover both of these embodiments, the example descriptions use the terms deep learning neural network, neural network, or NN interchangeably throughout.

### Example 1

### Building a Deep Learning Neural Network for Probability Distribution Predictions

Data Collection, Generation, and Cleaning: For experiments, high throughput methods were used to generate various genetic and environmental input conditions and one observation per input was collected. For simulations, randomly input combinations within valid ranges were generated followed by the running of stochastic simulations (e.g., Gillespie stochastic simulation algorithm or stochastic differential equation simulation) to obtain output observations. All inputs, whether from experimental or simulation data, were normalized to a standard scale. Where the observations were discrete numbers (e.g., mRNA counts, protein counts, etc.), the observations were represented by integers and the integers were included as one of the predicting points for the neural network outputs. Where the observations were continuous values (e.g., concentrations or optical density measurements), there were no non-applicable or infinity values and the observations were within the prediction range of the neural network.

Neural Network Construction: The input layer of the neural network was the intake for all of the input conditions and the output layer produced the probability distribution of the neural network. A negative log-likelihood algorithm was used as the loss function. Where the observations were discreet numbers, the neural network predicted the probability value for all possible discrete numbers and a neural network layer (e.g., a SOFTMAX® layer, Molecular Devices, LLC, San Jose, Calif., USA) was implemented to make sure that the sum for the predicted probability for any input condition equaled one. Where the observations were continuous values, the possible observation range was discretized into a reasonable number of bins (e.g., vertical bars on the graph, which represented the number of samples of the dataset). With the continuous values, the neural network predicted the probability value for the center of the bins and the probabilities for the remaining values were interpolated. The last layer was normalized with a normalization factor to make sure that the cumulative trapezoidal numerical integration of the probability distribution equaled one.

Neural Network Training: First, the neural network nodes were randomly initialized. Next, the output probability distribution was initialized to a uniform distribution and modified at each training epoch to minimize the loss of function. The gradient was then clipped to prevent exploration and the neural network was trained using a training data set consisting of the input conditions and one output observation. The performance of the trained neural network was tested with a small batch of the data that was not included in the training set. The probability distribution of this testing data was determined by repeated experiments and/or simulations.

Neural Network Predictions: After training, the trained neural network was ready to be used for predicting the probability distribution for any input genetic and environmental condition, such as facilitating the quantitative understanding of biological systems or designing synthetic gene circuits.

### Example 2

### Determining Probability Distributions for Theoretical mRNA Samples with Limited Observations

A deep learning neural network with a negative log-likelihood loss function was used to determine if the probability distribution of mRNA could be predicted with limited observation samples. The starting point for the analysis was the two-state model for stochastic gene transcription in single cells, which is shown schematically in FIG. 5. To train the neural network, the following input parameters were used: Kₒₙ, K_{off}, ν, and δ, where Kₒₙ represents a gene that is on and undergoing gene transcription; K_{off} represents a gene that is off and no undergoing gene transcription; ν is the transcription rate; and δ is the degradation rate. To minimize the loss function, the following n experimental observations were used for each parameter combination, n=1, 3, 10, 100, randomly drawn from the known distribution. As a control, the theoretical probability distribution of mean mRNA at a steady state, m, was calculated according to Formula (3):

$$p ( m ∨ {K}_{o n} , {K}_{off} , v , δ ) = \frac{1}{m !} \frac{Γ ( m + a ) Γ ( b )}{Γ ( m + b ) Γ ( a )} , \qquad ( 3 )$$

$$where$$

$$a = \frac{{K}_{o n}}{δ} ∧ b = \frac{{K}_{o n} + {K}_{off}}{δ}$$

Applying the neural network, six different theoretical probability distributions for mRNA were calculated where δ=1 and Kₒₙ, K_{off}, and ν have the following values: (1) Kₒₙ=K_{off}=0.01, ν=1; (2) Kₒₙ=K_{off}=0.1, ν=50; (3) Kₒₙ=K_{off}=0.5, ν=50; (4) Kₒₙ=K_{off}=1.0, ν=50; (5) Kₒₙ=K_{off}=1.2, ν=50; and (6) Kₒₙ=K_{off}=10, ν=50. The graphs for the six theoretical probability distributions for mRNA are shown in FIG. 6. Unlike graphs that are developed with assumptions, the graphs developed with the neural network do not adhere to a single predictable curve.

### Example 3

### Comparing Predicted Versus Real Probability Distributions for Theoretical mRNA Samples with Limited Observations

A deep learning neural network with a negative log-likelihood loss function was used to measure the probability distribution of the number of mRNA as a function of limited observations, n. The negative log-likelihood loss function of the NN was trained with the following limited observations: n=1, 3, 10, and 100. For implementation, the NN inputs were the values for Kₒₙ, K_{off}, ν, and δ, where δ=1, Kₒₙ, K_{off}, and ν are random chosen values, and Kₒₙ=K_{off}. The training set consisted of 10,000 data points and the test set consisted of 2000 data points. The probability distribution for the number of mRNA were the NN outputs. FIGS. 7A-7H show the results of the NN performance for each of the loss function training observations (n=1, 3, 10, and 100) compared to the real distribution values, the latter of which represent the real observations. While the NN models that were trained with the highest number of loss function observations (n=100) resulted in test set probability distributions that were almost identical to the real distributions, the NN models that were trained with the lower number of loss function observations (n=1, 3, and 10) produced results with high accuracy, including the model that was trained with a single observation (n=1). The results show that the probability distribution of mRNA can be accurately predicted with just one observation.

### Example 4

### Determining the Degree of Noise in Predicted Versus Real Probability Distributions for Theoretical mRNA Samples with Limited Observations

Because gene transcription is an inherently stochastic process, the ability of the NN (with the negative log-likelihood loss function) to overcome intrinsic noise and render accurate predictions for the probability distribution of the mRNA was tested by comparing, for each training observation, n=1, 3, 10, and 100, the predicted mean from the NN and the sample mean against real mean values. The sample mean values were calculated with Formula (3) as described in Example 2. As shown in FIGS. 8A and 8B, the predicted mean graphs (graphs on the right) for each observation, including the n=1 observation (FIG. 8A, top right), showed no discernable noise whereas the sample mean graphs (graphs on the left) for each observation showed noise increasing as the number of observations decreased with the n=1 observation sample mean graph showing a very high degree of noise (FIG. 8A, top left). As shown in FIGS. 8A and 8B, the negative log-likelihood loss function of the NN, even at a single training observation, eliminated the intrinsic noise present in the sample mean prediction by identifying and omitting outlier readings from the mean range. The result of the elimination of the outlier readings are mRNA probability distribution predictions that only include data points that are inherent to the mRNA data set.

### Example 5

### The Effect of Training Data Size on Probability Distribution Accuracy

To determine if the training data size affected the accuracy of the NN probability distribution predictions of Examples 3 and 4, the following nine different training data sizes were used to train the NN against a fixed size test set of 3000: 100, 200, 400, 800, 1600, 3200, 6400, 12800, and 25600. The results of the training size experiment are shown in FIG. 9 for three probability distribution tests, the loss function (mean negative log-likelihood) test and two control tests, a root mean square error test (RMSE) and a coefficient of determination test (R²). The RSME test was calculated according to Formula (4):

$$RSME = \sqrt{\frac{1}{n} {\sum }_{i = 1}^{n} {( {S}_{i} - {O}_{i} )}^{2}} , \qquad ( 4 )$$

where Oᵢ are the observations, Sᵢ are the predicted values of a variable, and n is the number of observations available for analysis. RMSE tests measure the accuracy of a prediction model by comparing prediction errors of different models or model configurations for a particular variable (but does not provide a comparison between variables). The R² test was calculated according to Formula (5):

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - {\overline{y}}_{i} )}^{2}} , \qquad ( 5 )$$

where y is an estimation of the average response and n is the number of points in the design of the experiments. The three tests were run for each of the nine training data sets after training with one observation (n=1) and separately after training with ten observations (n=10). As shown in FIG. 9, for all nine training data sets, the tests trained with the single observation yielded similar results to the tests trained with ten observations.

We claim:

1. A method of predicting probability distribution of a biological phenotype comprising:
gathering a data set comprising at least 3000 input parameters for a biological system and generating a limited data set comprising 1-10 output observations through experimentation and/or simulation of the input parameter data set;
building a deep learning neural network comprising a loss function and training the loss function with the limited data set of output observations; and
training the neural network with the input parameter data set, wherein output from the trained neural network comprises a predicted probability distribution of a biological phenotype associated with the biological system.

2. The method of claim 1, wherein the deep learning neural network is selected from a recurrent neural network and a long short-term memory network and the loss function is a negative log-likelihood function.

3. The method of claim 1, wherein the limited data set has a single observation.

4. The method of claim 1, wherein the predicted probability distribution is a continuous probability distribution of each input parameter for the biological system.

5. The method of claim 1, wherein the biological system is intrinsically noisy and the trained loss function calculates whether readings outside of the mean range of the input parameter data set are inherent to the biological system or outlier readings.

6. The method of claim 1, wherein the biological system is selected from the group consisting of a cellular system, a biological collective, a synthetic gene circuit, and combinations thereof.

7. The method of claim 1, wherein the input parameters are selected from the group consisting of cell growth rate, cell lysis rate, cell motility, gene expression, nutrient concentration, temperature, pH, activation rate, transcription rate, temperature, agar density, and combinations thereof.

8. The method of claim 1, wherein the biological phenotype is selected from the group consisting of number of mRNA produced, number of amino acids, number of proteins, cellular growth, cellular adhesion, cellular sensing, fluorescence strength, optical density, chemical concentration, and combinations thereof.

9. A method of predicting probability distribution of a biological phenotype comprising:
gathering a data set comprising at least 3000 input parameters for a biological system and generating a limited data set comprising 1-10 observations through experimentation and/or simulation of the input parameter data set;
building a recurrent neural network (RNN) comprising a negative log-likelihood loss function and training the negative log-likelihood loss function with the limited data set of output observations; and
training the RNN with the input parameter data set, wherein output from the trained RNN comprises a predicted probability distribution of a biological phenotype associated with the biological system.

10. The method of claim 9, wherein the predicted probability distribution is a continuous probability distribution of each input parameter for the biological system.

11. The method of claim 9, wherein the biological system is intrinsically noisy and the trained negative log-likelihood loss function calculates whether readings outside of the mean range of the input parameter data set are inherent to the biological system or outlier readings.

12. The method of claim 9, wherein the biological system is selected from the group consisting of a cellular system, a biological collective, a synthetic gene circuit, and combinations thereof.

13. The method of claim 9, wherein the input parameters are selected from the group consisting of cell growth rate, cell lysis rate, cell motility, gene expression, nutrient concentration, temperature, pH, activation rate, transcription rate, temperature, agar density, and combinations thereof.

14. The method of claim 9, wherein the biological phenotype is selected from the group consisting of number of mRNA produced, number of amino acids, number of proteins, cellular growth, cellular adhesion, cellular sensing, fluorescence strength, optical density, chemical concentration, and combinations thereof.

15. A method of predicting probability distribution of a biological phenotype comprising:
gathering a data set comprising at least 3000 input parameters for a biological system and generating a limited data set comprising 1-10 observations through experimentation and/or simulation of the input parameter data set;
building a long short-term memory (LSTM) network comprising a negative log-likelihood loss function and training the negative log-likelihood loss function with the limited data set of output observations; and
training the LSTM network with the input parameter data set, wherein output from the trained LSTM network comprises a predicted probability distribution of a biological phenotype associated with the biological system.

16. The method of claim 15, wherein the predicted probability distribution is a continuous probability distribution of each input parameter for the biological system.

17. The method of claim 15, wherein the biological system is intrinsically noisy and the trained negative log-likelihood loss function calculates whether readings outside of the mean range of the input parameter data set of the input parameters are inherent to the biological system or outlier readings.

18. The method of claim 15, wherein the biological system is selected from the group consisting of a cellular system, a biological collective, a synthetic gene circuit, and combinations thereof.

19. The method of claim 15, wherein the input parameters are selected from the group consisting of cell growth rate, cell lysis rate, cell motility, gene expression, nutrient concentration, temperature, pH, activation rate, transcription rate, temperature, agar density, and combinations thereof.

20. The method of claim 15, wherein the biological phenotype is selected from the group consisting of number of mRNA produced, number of amino acids, number of proteins, cellular growth, cellular adhesion, cellular sensing, fluorescence strength, optical density, chemical concentration, and combinations thereof.
